(19) United States
(12) Patent Application Publication
(10) Pub. No.: US 2020/0102591 A1
(43) Pub. Date: Apr. 2, 2020

(54) Methods for the Imaging of Nucleic Acid Sequences

(71) Applicant: President and Fellows of Harvard College, Cambridge, MA (US)

(72) Inventor: Chao-ting Wu, Brookline, MA (US)

(21) Appl. No.: 16/498,495

(22) PCT Filed: Mar. 30, 2018
(86) PCT No.: PCT/US18/25409

§ 371 Date: Sep. 27, 2019

Related U.S. Application Data
(60) Provisional application No. 62/479,783, filed on Mar. 31, 2017.

(51) Int. Cl. C12Q 1/682

(52) U.S. Cl. CPC C12Q 1/682; C12Q 2525/117; C12Q 2525/161; C12Q 2565/601; C12Q 2563/113; C12Q 2565/102; C12Q 2543/10

(57) ABSTRACT

The present invention relates to methods of providing sequence specificity to in situ genome imaging at the level of the electron microscope (EM).

### RELATED APPLICATION DATA

This application claims priority to U.S. Provisional Application No. 62/479,783 filed on Mar. 31, 2017, which is hereby incorporated herein by reference in its entirety for all purposes.

### STATEMENT OF GOVERNMENT INTERESTS

This invention was made with government support under grant number DP1GM106412 awarded by the NIH, grant number RM1HG008525 awarded by the NIH, and grant number R01HD091797 awarded by the NIH. The government has certain rights in the invention.

### FIELD

The present disclosure is directed to imaging target nucleic acid sequences, such as in vivo target nucleic acid sequences.

### BACKGROUND

Electron microscope technologies exist for imaging genomic DNA. However, current technologies that utilize an electron microscope for the in situ imaging of the genome cannot easily differentiate between one region of the genome from another in a sequence-specific fashion. Accordingly, methods are required for the sequence-specific in situ imaging of genomic nucleic acid sequences.

### SUMMARY

The present disclosure relates in general to improving the detectability or visibility of a target nucleic acid, such as a target nucleic acid sequence in situ. The present disclosure relates in general to improving the performance or capability of an electron microscope to visualize or detect a target nucleic acid, such as a target nucleic acid sequence in situ by delivering a detectable moiety to the target nucleic or by making or rendering the target nucleic acid more detectable compared to the naked or naturally occurring target nucleic acid. According to certain aspects, oligopaint technology is combined with electron microscope technology. Oligopaint technology is generally known in the art. Oligopaints are used to hybridize to a target nucleic acid sequence in situ. According to one aspect, oligopaints are used to hybridize to a target nucleic acid sequence and to deliver a functional moiety to a target nucleic acid sequence. The functional moiety may be delivered directly or indirectly to the target nucleic acid sequence. The functional moiety may directly assist in the visualization of the target nucleic acid sequence using the electron microscope insofar as the functional moiety is a detectable moiety, i.e., one which can be detected by an electron microscope. The functional moiety may facilitate the detection of the target nucleic acid sequence by providing a particular function which results in a detectable target nucleic acid sequence. For example, a functional moiety may be a polymerization initiator which facilitates polymerization of monomers at or near the target nucleic acid sequence to produce a polymer which may facilitate viewing of the target nucleic acid sequence or which polymer may be treated or stained with a detectable moiety, such as an electron dense compound, to facilitate viewing of the target nucleic acid sequence by the electron microscope.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a schematic representation of a probe system utilizing nongenomic Mainstreet and Backstreet sequences flanking a genomic sequence. The genomic sequence can be DNA, RNA, a DNA/RNA hybrid or a gRNA, such as is used with a CRISPR system.

FIG. 2 is a schematic representation DAB-based EM using the photogeneration of single oxygen by DNA bound dye that leads to deposition of DAB polymers, which produce contrast when bound by OsO₄.

FIG. 3 is a schematic representation of sequence-specific EM-imaging of the genome that can be achieved by targeting labeled Oligopaint probes to specific genomic sites.

### DETAILED DESCRIPTION

The present disclosure is directed to imaging target nucleic acid sequences, such as those in vivo, using oligopaints as are generally known in the art, to deliver functional moieties directly or indirectly to the target nucleic acid sequences, which are useful in the viewing of the target nucleic acid sequences, such as by using electron microscope technology. According to one aspect, oligopaints designed to be complementary to target nucleic acid sequences such as genomic sequences provide specificity of delivery of functional moieties to the target nucleic acid sequence, such as in vivo target nucleic acid sequences, for imaging. This is referred to as sequence specificity insofar as a target sequence is labeled with a functional moiety.

According to one aspect, functional moieties may include electron dense moieties which may be conjugated or otherwise attached to oligopaints and the oligopaints are then hybridized to a target nucleic acid sequence, such as a genomic DNA. Once the oligopaints are hybridized, the electron dense moieties attached thereto may be imaged or identified by electron microscope technology. In this manner, the structure of the target nucleic acid sequence which has been labelled with electron dense moieties, i.e. one or more targeted genomic regions, can be elucidated or otherwise determined.

According to one aspect, functional moieties may include oligonucleotide nanostructures, such as DNA origami, which themselves may be visualized or imaged or which may include or bind to other moieties which facilitate visualization or imaging. Once the oligopaints are hybridized, the oligonucleotide nanostructures may be imaged or identified, such as by electron microscope technology. In this manner, the structure of the target nucleic acid sequence which has been labelled with oligonucleotide nanostructures, i.e. one or more targeted genomic regions, can be elucidated or otherwise determined. Imaging can be enhanced by attaching or adding additional functional or detectable moieties to the oligonucleotide nanostructures.

According to one aspect, functional moieties may include polymerization initiators which facilitate polymerization of monomers into polymers to produce a polymer localized at the target nucleic acid sequence. Once the oligopaints are hybridized, the polymerization initiators initiate polymerization of nearby monomers to produce a localized polymer which may be imaged or identified, such as by electron microscope technology. In this manner, the structure of the target nucleic acid sequence which has been labelled with a localized polymer, i.e. one or more targeted genomic regions, can be elucidated or otherwise determined. Imaging can be enhanced by attaching or adding additional functional or detectable moieties to the localized polymer.

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, Molecular Cloning: A Laboratory Manual, Second Edition (1989), Oligonucleotide Synthesis (M. J. Gait Ed., 1984), Animal Cell Culture (R. I. Freshney, Ed., 1987), the series Methods in Enzymology (Academic Press, Inc.); Gene Transfer Vectors for Mammalian Cells (J. M. Miller and M. P. Calos eds. 1987), Handbook of Experimental Immunology, (D. M. Weir and C. C. Blackwell, Eds.), Current Protocols in Molecular Biology (F. M. Ausubel, R. Brent, R. E. Kingston, D. D. Moore, J. G. Siedman, J. A. Smith, and K. Struhl, eds., 1987), Current Protocols in Immunology (J. E. coligan, A. M. Kruisbeek, D. H. Margulies, E. M. Shevach and W. Strober, eds., 1991); Annual Review of Immunology; as well as monographs in journals such as Advances in Immunology. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Komberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

It is to be understood that method steps described herein need not be performed in the order listed unless expressly stated. Method steps may be performed in any order. Further, method steps may be performed simultaneously or together and need not be performed separately or individually. To the extent that methods describe multiple oligopaints being hybridized to various target nucleic acid sequences, such hybridization may be performed as a single step with all reagents combined. Individual hybridization steps need not be performed individually.

The present disclosure provides a method of imaging a target nucleic acid sequence in situ. The method includes hybridizing a plurality of oligopaints to the target nucleic acid sequence. Each oligopaint of the plurality includes a complementary nucleic acid sequence and a first non-genomic nucleic acid sequence. The first non-genomic nucleic acid sequence includes a polymerization initiator attached thereto. The polymerization initiator is activated in the presence of monomers to initiate polymerization of the monomers to create a polymer fixed, connected, or otherwise complexed with the target nucleic acid sequence. The target nucleic acid sequence with the polymer fixed thereto is then imaged using a suitable electron microscope technology.

### Oligopaints

The disclosure provides probes which may be oligonucleotide or polynucleotide probes. Such oligonucleotide or polynucleotide probes may be referred to as Oligopaint probes or Oligopaints or chromosome paints as is known in the art. See US-2010-0304994 hereby incorporated by reference in its entirety. Oligopaints are computationally designed single-stranded DNA oligonucleotide probes that can be used to visualize genomic regions as small as a few kilobases (kbs) to as large as tens of megabases (Mbs) using conventional, confocal, or super-resolution microscopy. Nucleic acid sequences or oligonucleotide probes according to the present disclosure may have any desired length. The term “probe” refers to a single-stranded oligonucleotide sequence that will recognize and form a hydrogen-bonded duplex with a complementary sequence in a target nucleic acid sequence or its cDNA derivative. The probe includes a target hybridizing nucleic acid sequence. A probe provided by the disclosure includes a complementary sequence complementary to a strand of the target genomic nucleic acid sequence. Oligonucleotide or polynucleotide probes or oligopaints may be designed, if desired, with the aid of a computer program such as, for example, DNAWorks, or Gene2Oligo. Oligopaints are described in US2014/0364333 hereby incorporated by reference in its entirety.

The complementary sequence may have a nucleotide length between about 15 and about 1000 bases. The complementary sequence may have a nucleotide length between about 15 and about 500 bases. The complementary sequence may have a nucleotide length between about 15 and about 400 bases. The complementary sequence may have a nucleotide length between about 15 and about 300 bases. The complementary sequence may have a nucleotide length between about 15 and about 200 bases. The complementary sequence may have a nucleotide length between about 15 and about 100 bases. The complementary sequence may have a nucleotide length between about 15 and about 90 bases. The complementary sequence may have a nucleotide length between about 15 and about 80 bases. The complementary sequence may have a nucleotide length between about 15 and about 70 bases. The complementary sequence may have a nucleotide length between about 15 and about 60 bases. The complementary sequence may have a nucleotide length between about 15 and about 50 bases. The complementary sequence may have a nucleotide length between about 15 and about 40 bases. The complementary sequence may have a nucleotide length between about 15 and about 30 bases. The complementary sequence may have a nucleotide length between about 20 and about 1000 bases. The complementary sequence may have a nucleotide length between about 20 and about 500 bases. The complementary sequence may have a nucleotide length between about 20 and about 100 bases. The complementary sequence may have a nucleotide length between about 20 and about 80 bases. The complementary sequence may have a nucleotide length between about 20 and about 40 bases. The complementary sequence may have a nucleotide length between about 20 and about 100 bases. The complementary sequence may have a nucleotide length between about 20 and about 60 bases. The complementary sequence may have a nucleotide length of about 22, 32, 40, 50 or 60 bases.

Oligopaints have a high resolution useful in detecting and identifying target genomic nucleic acids. As used herein, the term “resolution” refers to the ability to distinguish (e.g., label) between two points on a polynucleotide sequence (e.g., two points along the length of a chromosome). As used herein, the term “high resolution” refers to the ability to detect two or more nucleic acid sequences having a distance of less than 6×10⁶ base pairs apart (e.g., on a chromosome). In certain aspects, two or more high resolution Oligopaints have a resolution of about 500 kilobases apart or fewer, 400 kilobases apart or fewer, 300 kilobases apart or fewer, 200 kilobases apart or fewer, 100 kilobases apart or fewer, 90 kilobases apart or fewer, 80 kilobases apart or fewer, 70 kilobases apart or fewer, 60 kilobases apart or fewer, 50 kilobases apart or fewer, 40 kilobases apart or fewer, 30 kilobases apart or fewer, 20 kilobases apart or fewer, 19 kilobases apart or fewer, 18 kilobases apart or fewer, 17 kilobases apart or fewer, 16 kilobases apart or fewer, 15 kilobases apart or fewer, 14 kilobases apart or fewer, 13 kilobases apart or fewer, 12 kilobases apart or fewer, 11 kilobases apart or fewer, 10 kilobases apart or fewer, 9 kilobases apart or fewer, 8 kilobases apart or fewer, 7 kilobases apart or fewer, 6 kilobases apart or fewer, 5 kilobases apart or fewer, 4 kilobases apart or fewer, 3 kilobases apart or fewer, 2 kilobases apart or fewer or 1 kilobase apart or fewer. In certain aspects, two or more high resolution Oligopaints have a resolution of about 1900 bases apart or fewer, 1800 bases apart or fewer, 1700 bases apart or fewer, 1600 bases apart or fewer, 1500 bases apart or fewer, 1400 bases apart or fewer, 1300 bases apart or fewer, 1200 bases apart or fewer, 1100 bases apart or fewer, 1000 bases apart or fewer, 900 bases apart or fewer, 800 bases apart or fewer, 700 bases apart or fewer, 600 bases apart or fewer, 500 bases apart or fewer, 400 bases apart or fewer, 300 bases apart or fewer, 200 bases apart or fewer, 100 bases apart or fewer, 95 bases apart or fewer, 90 bases apart or fewer, 85 bases apart or fewer, 80 bases apart or fewer, 75 bases apart or fewer, 70 bases apart or fewer, 65 bases apart or fewer, 60 bases apart or fewer, 55 bases apart or fewer, 50 bases apart or fewer, 45 bases apart or fewer, 40 bases apart or fewer, 35 bases apart or fewer, 30 bases apart or fewer, 25 bases apart or fewer, 20 bases apart or fewer, 15 bases apart or fewer, 10 bases apart or fewer or down to the individual base pair. In certain aspects, two or more high resolution Oligopaints have a resolution of between about 10 bases and about 2000 bases, between about 10 bases and about 1000 bases, between about 10 bases and about 500 bases, between about 15 bases and about 250 bases, between about 15 bases and about 100 bases, between about 20 bases and about 50 bases, or between about 20 bases and about 30 bases.

As used herein, the term “sensitivity,” with respect to probes, refers to the number of target nucleotide bases (e.g., target genomic nucleotide bases) that are complementary to a particular probe, i.e., the number of target nucleotide bases to which a particular probe can hybridize (i.e., the smallest band size that can be detected). In certain aspects, high resolution probes have a resolution of about 1 kilobase, about 1900 bases, about 1800 bases, about 1700 bases, about 1600 bases apart, about 1500 bases, about 1400 bases, about 1300 bases, about 1200 bases, about 1100 bases, about 1000 bases, about 900 bases, about 800 bases, about 700 bases, about 600 bases, about 500 bases, about 400 bases, about 300 bases, about 200 bases, about 100 bases, about 95 bases, about 90 bases, about 85 bases, about 80 bases, about 75 bases, about 70 bases, about 65 bases, about 60 bases, about 55 bases, about 50 bases, about 45 bases, about 40 bases, about 35 bases, about 30 bases, about 25 bases, about 20 bases, about 15 bases, about 10 bases, or about 5 bases. In certain aspects, the number of target nucleotide bases that are complementary to a probe are consecutive (e.g., consecutive genomic nucleotide bases).

According to one aspect, the disclosure provides for the use of oligopaints having a complementary sequence between about 5 bases and about 100 bases, between about 5 bases and about 95 bases, between about 5 bases and about 90 bases, between about 5 bases and about 85 bases, between about 5 bases and about 80 bases, between about 5 bases and about 75 bases, between about 5 bases and about 70 bases, between about 5 bases and about 65 bases, between about 5 bases and about 60 bases, between about 5 bases and about 55 bases, between about 5 bases and about 50 bases, between about 5 bases and about 45 bases, between about 5 bases and about 40 bases, between about 5 bases and about 35 bases, between about 5 bases and about 30 bases, between about 5 bases and about 25 bases, between about 5 bases and about 20 bases, between about 5 bases and about 15 bases, between about 5 bases and about 10 bases, between about 15 bases and about 50 bases, and between about 20 bases and about 40 bases.

Oligopaints with such nucleotide lengths can access targets that are not accessible to longer oligonucleotide probes. For example, in certain aspects, small oligopaints can pass into a cell, can pass into a nucleus, and/or can hybridize with targets that are partially bound by one or more proteins, etc. Small probes are also useful for reducing background, as they can be more easily washed away than larger hybridized oligonucleotide sequences.

The disclosure provides the design and use of multiple oligopaints that hybridize to a target genomic locus to create a combined signal which can be used to detect and identify the target genomic locus. As an example, a plurality or set or library of DNA oligonucleotide paint probes are designed such that a number of DNA oligonucleotide paint probes are used to hybridize to a genomic locus, such that the probes generate a combined signal with enhanced photon yield and signal-to-noise ratio.

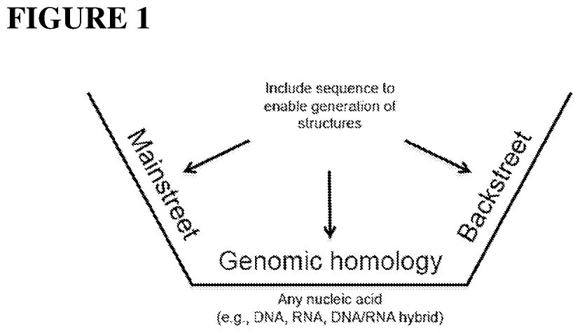

In general and with reference to FIG. 1, an oligopaint includes a complementary nucleic acid sequence that is complementary to a target oligonucleotide sequence, such as a portion of a DNA sequence, or a particular chromosome or sub-chromosomal region of a particular chromosome. The complementary nucleic acid sequence may be said to have genomic homology insofar as the oligopaint is intended to hybridize with a complementary genomic nucleic acid sequence. The complementary nucleic acid sequence may be between 15 to 50 or between 32 to 42 bases in length. The complementary nucleic acid sequence may by any nucleic acid sequence and may be a DNA sequence, an RNA sequence (such as a guide RNA sequence as is understood with CRISPR systems) or a DNA/RNA hybrid sequence. The oligopaint may also include a nongenomic nucleic acid sequence or region upstream of the complementary nucleic acid sequence which may be referred to as a “Mainstreet” sequence. The oligopaint may also include a nongenomic nucleic acid sequence or region downstream of the complementary nucleic acid sequence which may be referred to as a “Backstreet” sequence. The oligopaint may include both a first nongenomic nucleic acid sequence or region upstream of the complementary nucleic acid sequence (“Mainstreet”) and a second nongenomic nucleic acid sequence or region downstream of the complementary nucleic acid sequence (“Backstreet”). In this manner the complementary or genomic nucleic acid sequence may be flanked by a Mainstreet sequence and a Backstreet sequence. While the purpose of the complementary or genomic nucleic acid sequence is to hybridize with a target genomic nucleic acid sequence, the Mainstreet and Backstreet sequences may be used to carry functional moieties. The functional moieties may be directly attached to the Mainstreet or Backstreet sequences or they may be indirectly attached to the Mainstreet or Backstreet sequences. For example, a functional moiety may be indirectly attached insofar as the functional moiety is directly attached to a first nongenomic nucleic acid sequence probe which is complementary to a portion of the nongenomic Mainstreet or Backstreet sequences. In this manner, the first nongenomic nucleic acid sequence probe hybridizes to the complementary portion of the nongenomic Mainstreet or Backstreet sequences.

In general, a plurality or set or library of nucleic acid oligopaint probes, such as DNA oligonucleotides, may be synthesized using a DNA microarray, or a DNA chip. The oligonucleotides may contain one or more sequences used for the purpose of amplification by polymerase chain reaction (PCR), in vitro transcription (IVT), and other biochemical processing steps such as adding additional sequence by ligation or polymerization, single-stranding, and processing by restriction enzymes, in order to generate a final library of oligonucleotides. Such methods are known to those of skill in the art and are described in U.S. Pat. No. 9,476,089, US 2012-0295801 and US 2010-0304994 each of which are hereby incorporated by reference in its entirety.

Probes, such as oligopaint probes, may be generated from synthetic probes and arrays that are, optionally, computationally patterned (rather than using natural DNA sequences and/or chromosomes as a template). Probes may be made by any suitable method including array based methods as described in US 2010-0304994. Such a method includes the steps of providing at least one solid support having a plurality of synthetic, single stranded oligonucleotide sequences attached thereto wherein a portion of each of the plurality of synthetic, single stranded oligonucleotide sequences is complementary to a portion of a specific chromosome sequence, synthesizing a plurality of complementary strands, each of which is complementary to a synthetic, single stranded oligonucleotide sequence attached to the at least one solid support, removing the plurality of complementary strands from the at least one solid support, and optionally amplifying the plurality of complementary strands to produce a set of oligonucleotide paints. Oligopaints or oligonucleotide paints, have a resolution of about two kilobases or fewer. In certain aspects, each probe has a resolution of about one kilobase or fewer or 100 bases or fewer. In certain aspects, the set of probes has a resolution of between about 20 bases and about 30 bases.

The disclosure provides that synthesis of oligopaints and/or amplification of oligopaints can be performed using a support. As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. However, synthetic or non-natural nucleotides may be used. In certain aspects, multiple supports (tens, hundreds, thousands or more) may be utilized (e.g., synthesized, amplified, hybridized or the like) in parallel. Suitable supports include, but are not limited to, slides (e.g., microscope slides), beads, chips, particles, strands, gels, sheets, tubing (e.g., microfuge tubes, test tubes, cuvettes), spheres, containers, capillaries, microfibers, pads, slices, films, plates (e.g., multi-well plates), microfluidic supports (e.g., microarray chips, flow channel plates, biochips and the like) and the like. In various embodiments, the solid supports may be biological, nonbiological, organic, inorganic or combinations thereof. When using supports that are substantially planar, the support may be physically separated into regions, for example, with trenches, grooves, wells, or chemical barriers (e.g., lacking a lipid-binding coating). In exemplary embodiments, supports can be made of a variety of materials including, but not limited to glass, quartz, ceramic, plastic, polystyrene, methylstyrene, acrylic polymers, titanium, latex, sepharose, cellulose, nylon and the like and any combination thereof. Such supports and their uses are well known in the art. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. When using a support that is substantially planar, the support may be physically separated into regions, for example, with trenches, grooves, wells, or chemical barriers (e.g., hydrophobic coatings, etc.).

In certain exemplary embodiments, a support is a microarray. As used herein, the term “microarray” refers in one embodiment to a type of assay that comprises a solid phase support having a substantially planar surface on which there is an array of spatially defined non-overlapping regions or sites that each contain an immobilized nucleic acid such as a hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate create a substantially planar surface. Spatially defined sites may additionally be “addressable” in that its location and the identity of the immobilized probe at that location are known or determinable.

Oligonucleotide sequences useful as probes may be prepared by any suitable method, e.g., the phosphoramidite method described by Beaucage and Carruthers ((1981) Tetrahedron Lett. 22: 1859) or the triester method according to Matteucci et al. (1981) J. Am. Chem. Soc. 103:3185), both incorporated herein by reference in their entirety for all purposes, or by other chemical methods using either a commercial automated oligonucleotide synthesizer or high-throughput, high-density array methods described herein and known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides and chips containing oligonucleotides may also be obtained commercially from a variety of vendors.

In an exemplary embodiment, oligopaints may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. An exemplary type of instrument is the type shown in FIG. 5 of U.S. Pat. No. 6,375,903, based on the use of reflective optics. Other methods for synthesizing oligonucleotide probes (e.g., Oligopaints) include, for example, light-directed methods utilizing masks, flow channel methods, spotting methods, pin-based methods, and methods utilizing multiple supports as is known in the art. In yet another embodiment, a plurality of oligonucleotide probes (e.g., Oligopaints) may be synthesized on multiple supports. One example is a bead based synthesis method which is described, for example, in U.S. Pat. Nos. 5,770,358, 5,639,603, and 5,541,061.

In one embodiment, oligonucleotide probes synthesized on a solid support may be used as a template for the production of oligonucleotide probes, such as oligopaints. For example, the support bound oligonucleotides may be contacted with primers that hybridize to the oligonucleotides under conditions that permit chain extension of the primers. The support bound duplexes may then be denatured and pooled and used as oligopaints or they may be subjected to further rounds of amplification to produce the probes, such as Oligopaints, in solution. In another embodiment, the support-bound oligonucleotide probes may be removed from the solid, pooled and amplified to produce probes, i.e. Oligopaints, in solution. The oligonucleotides may be removed from the solid support, for example, by exposure to conditions such as acid, base, oxidation, reduction, heat, light, metal ion catalysis, displacement or elimination chemistry, or by enzymatic cleavage.

In various embodiments, the methods disclosed herein comprise amplification of oligonucleotide sequences, i.e., probes, including oligopaints. Amplification methods may comprise contacting a nucleic acid, such as an oligopaint, with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art.

In general, high resolution oligonucleotide paints may be made by computationally determining genomic spacing of a plurality of synthetic, oligonucleotide sequences, wherein each of the plurality is complementary to a portion of a specific chromosome sequence, synthesizing the plurality of synthetic oligonucleotide sequences, and adding a functional moiety if desired to produce a plurality of oligonucleotide paints, wherein the set of oligonucleotide paints has a resolution of about two kilobases or fewer, and wherein each of a plurality of the oligonucleotide paints is complementary to a target nucleic acid sequence (e.g., a genomic sequence), such as of 40 consecutive nucleotide bases or fewer. Certain exemplary embodiments are directed to the use of computer software to automate design and/or interpretation of genomic spacings, complementary sequences and barcode sequences for each specific set of oligonucleotides or oligopaints. Such software may be used in conjunction with individuals performing interpretation by hand or in a semi-automated fashion or combined with an automated system. In at least some embodiments, the design and/or interpretation software is implemented in a program written in the JAVA programming language. The program may be compiled into an executable that may then be run from a command prompt in the WINDOWS XP operating system. Unless specifically set forth in the claims, the invention is not limited to implementation using a specific programming language, operating system environment or hardware platform.

Hybridization of the oligopaints of the disclosure to target nucleic acid sequences such as chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized oligopaints). The reagents used in each of these steps and their conditions of use vary depending on the particular situation. For instance, step 3 will not always be necessary as the probes described herein can be designed to avoid repetitive sequences. Hybridization conditions are also described in U.S. Pat. No. 5,447,841. It will be appreciated that numerous variations of in situ hybridization protocols and conditions are known and may be used in conjunction with the present invention by practitioners following the guidance provided herein. In this manner, the target nucleic acid sequence may be separated into an upper strand and a lower strand, and the oligopaint is hybridized to the upper strand or the lower strand.

As used herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” Oligonucleotide probes according to the present disclosure need not form a perfectly matched duplex with the single stranded nucleic acid, though a perfect matched duplex is exemplary. According to one aspect, oligonucleotide probes as described herein form a stable hybrid with that of the target sequence under stringent to moderately stringent hybridization and wash conditions. If it is expected that the probes will be essentially completely complementary (i.e., about 99% or greater) to the target sequence, stringent conditions will be used. If some mismatching is expected, with the result that the probe will not be completely complementary, the stringency of hybridization may be lessened. “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tr for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5× SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA. It is to be understood that any desired stringency and/or conditions may be employed as desired.

### Target Nucleic Acid Sequences

The terms “nucleic acid,” “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), cDNA, recombinant polynucleotides, branched polynucleotides, plasmids, vectors, isolated DNA of a sequence, isolated RNA of a sequence, nucleic acid probes, and primers. Oligonucleotides or polynucleotides useful in the methods described herein may comprise natural nucleic acid sequences and variants thereof, artificial nucleic acid sequences, or a combination of such sequences. Oligonucleotides or polynucleotides may be single stranded or double stranded.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

According to certain aspects, a target nucleic acid sequence is any sequence to which it is desired to hybridize, such as by in situ hybridization, one or more oligopaints, such as for improving visualization or detection. The target nucleic acid sequence may be in vivo, i.e. in situ, or ex vivo. The target nucleic acid sequence may be DNA, genomic DNA, chromosomal DNA, RNA, single-copy DNA, repeated DNA, in situ DNA, in vitro DNA, cDNA, synthetic DNA, antibodies with a nucleic acid tail, or combinations thereof.

The in situ hybridization methods described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. The cells may be live cells. Samples are prepared for assays of the invention using conventional techniques, which typically depend on the source from which a sample or specimen is taken. These examples are not to be construed as limiting the sample types applicable to the methods and/or compositions described herein.

The disclosure provides for the hybridization of oligopaint probes to a target nucleic acid sequence, such as a target genomic nucleic acid sequence, where the oligopaint probes have a functional moiety attached thereto. The target genomic nucleic acid sequence may be a genomic locus. The size of the genomic locus may be between 100 bp and the whole genome. Exemplary lengths include that of a single histone (about 100-200 bp), a single gene (about 1-3 kb), a 1-2 Mb region of the genome, an arm of a chromosome (100 to 600 Mb) a single chromosome (100-1000 Mb), a whole genome (on the order of 1-2 Gb) (such as for distinguishing between whole bacterial genomes). One aspect of the present disclosure provides for the sequence specific direction of a functional moiety to a particular target nucleic acid sequence for visualization using an electron microscope technology. “Sequence specific” may refer to the labelling of a certain target portion of a larger nucleic acid sequence, such as to detect the certain target portion that has been labeled.

The disclosure provides that the target nucleic acid sequence may be a genomic nucleic acid sequence or region of a genomic nucleic acid, such as a chromosome or a sub-chromosomal region. The oligopaint probes described herein can be used to detect and identify chromosomes and sub-chromosomal regions of chromosomes during various phases of the cell cycle including, but not limited to, interphase, preprophase, prophase, prometaphase, metaphase, anaphase, telophase and cytokenesis.

As used herein, the term “chromosome” refers to the support for the genes carrying heredity in a living cell, including DNA, protein, RNA and other associated factors. The conventional international system for identifying and numbering the chromosomes of the human genome is used herein. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

As used herein, the term “chromosome banding” refers to differential staining of chromosomes resulting in a pattern of transverse bands of distinguishable (e.g., differently or alternately colored) regions, that is characteristic for the individual chromosome or chromosome region (i.e., the “banding pattern”). Conventional banding techniques include G-banding (Giemsa stain), Q-banding (Quinacrine mustard stain), R-banding (reverse-Giemsa), and C-banding (centromere banding).

As used herein, the term “karyotype” refers to the chromosome characteristics of an individual cell, cell line or genome of a given species, as defined by both the number and morphology of the chromosomes. Karyotype can refer to a variety of chromosomal rearrangements including, but not limited to, translocations, insertional translocations, inversions, deletions, duplications, transpositions, anueploidies, complex rearrangements, telomere loss and the like. Typically, the karyotype is presented as a systematized array of prophase or metaphase (or otherwise condensed) chromosomes from a photomicrograph or computer-generated image. Interphase chromosomes may also be examined

As used herein, the terms “chromosomal aberration” or “chromosome abnormality” refer to a deviation between the structure of the subject chromosome or karyotype and a normal (i.e., non-aberrant) homologous chromosome or karyotype. The deviation may be of a single base pair or of many base pairs. The terms “normal” or “non-aberrant,” when referring to chromosomes or karyotypes, refer to the karyotype or banding pattern found in healthy individuals of a particular species and gender. Chromosome abnormalities can be numerical or structural in nature, and include, but are not limited to, aneuploidy, polyploidy, inversion, translocation, deletion, duplication and the like. Chromosome abnormalities may be correlated with the presence of a pathological condition or with a predisposition to developing a pathological condition. Chromosome aberrations and/or abnormalities can also refer to changes that are not associated with a disease, disorder and/or a phenotypic change. Such aberrations and/or abnormalities can be rare or present at a low frequency (e.g., a few percent of the population (e.g., polymorphic)).

The disclosure provides that the target genomic nucleic acid sequence, such as DNA, can be inside the cell or on a substrate, such as glass, for example as by a “metaphase spread” technique where chromosomes are arrayed on a slide, which is common for karyotyping. The DNA could be in a natural or artificial conformation, e.g. stretched within a flow cell.

### Polymerization Initiators and Monomers

According to one aspect, methods of the present disclosure utilize a polymerization initiator attached to an oligopaint to initiate polymerization of monomers to form a polymer complexed with the target nucleic acid sequence. In this manner, the polymer may be said to be deposited on the target nucleic acid sequence. According to this aspect, the polymerization initiator is directly or indirectly attached to the oligopaint. The polymerization initiator may be directly or indirectly attached to the oligopaint by being directly or indirectly attached to a nongenomic nucleic acid sequence present as part of the oligopaint. As described herein, the nongenonic nucleic acid sequence may be upstream or downstream of a genomic nucleic acid sequence or the genomic nucleic acid sequence may be flanked by nongenomic nucleic acid sequences. The polymerization initiator may be attached to either the upstream nongenomic nucleic acid sequence or the downstream nongenomic nucleic acid sequence.

Exemplary polymerization initiators are known to those of skill in the art. When the oligopaint is hybridized to the target nucleic acid sequence, the polymerization initiator may then be activated using methods known to those of skill in the art for the particular polymerization initiator that is selected.

According to one aspect, the polymerization initiator is a singlet oxygen generator. According to one aspect, the polymerization initiator is a photoinduced polymerization initiator. According to one aspect, the polymerization initiator is a photoinduced polymerization initiator and polymerization is induced with a laser. According to one aspect, the polymerization initiator generates oxygen singlets to induce polymerization of the monomers. According to one aspect, the polymerization initiator is a dye or a fluorophore. According to one aspect, an exemplary polymerization initiator is a dye or a fluorophore, such as fluorescein, dibromofluorescein (DBF), eosin, tetramethylrhodamine (TAMRA), monobromo-TAMRA (Br-TAMRA), AlexaFluor 488 (AF488), AlexaFluor 633 (AF633), monobromo-Cy5 (Br-Cy5), methylene blue (MB), or IRDye700DX.

Exemplary monomers or oligomers to form polymers are known to those of skill in the art and include those which polymerize through initiation by singlet oxygen, such as aromatic amino monomers. An exemplary monomer is 3,3′-diaminobenzidine or DAB. Exemplary monomers may include diphenyl isophthalate, isophthalic acid, pyridine dicarboxylic acid, 2,6-dicarboxynaphthalene, 2,6-dicarboxypyridine, 2,5-dihydroxyterephthalic acid, 5-sulfoisophthalic acid, 2,3-bis(4-carboxylphenyl)quinoxalin, 4,6-dihydroxyisophthalic acid, 2,6-naphthalenedicarboxylic acid, 4-trifluoromethylphthalic acid, 4,4′-stilbenedicarboxylic acid, 3,3′,4,4′-tetraaminobiphenyl, 1,2,4,5-tetraaminobenzene, 2,3,5,6-tetraaminotoluene, 4,6-diaminoresorcinol, 2,5-diaminohydroquinone, 2,5-diamino-1,4-dithiobenzene, 3,3′-diaminobenzidine, 4,4′-diamino-2-phenylbiphenyl, 3,3′,4,4′-tetraaminobenzophenone, 3,3′,4,4′-tetraaminodiphenylmethane, 3,4-diaminobenzoic acid, 2,5-diaminobenzenesulfonic acid, or 5-(N-phthalimide)isophthalic acid.

Exemplary polymers include those formed by the polymerization of 3,3′-diaminobenzidine monomers forming a 3,3′-diaminobenzidine polymer. Exemplary polymers include poly(benzimidazole) or poly(benzobisimidazole) and the like. Exemplary polymer may include those formed by polymerization of the monomers listed herein.

A representative polymerization approach is described in Ngo J T, Adams S R, Deerinck T J, Boassa D, Rodriguez-Rivera F, Palida S F, Bertozzi C R, Ellisman M H, Tsien R Y. Click-EM for imaging metabolically tagged nonprotein biomolecules. Nat Chem Biol. 2016 12:459-65. doi: 10.1038/nchembio.2076. Epub 2016 Apr. 25.PMID:27110681 PMCID: PMC4871776 and Ou, 2015 Meth 90:39, 29 each of which is hereby incorporated by reference in its entirety.

### Electron Microscopy

Methods of imaging target genomic DNA, such as that with a detectable moiety such as a polymer or detectable particle, are known to those of skill in the art and include electron microscope technology. An electron microscope is a microscope that uses a beam of accelerated electrons as a source of illumination. As the wavelength of an electron can be up to 100,000 times shorter than that of visible light photons, electron microscopes have a higher resolving power than light microscopes and can reveal the structure of smaller objects. A transmission electron microscope can achieve better than 50 μm resolution and magnifications of up to about 10,000,000× whereas most light microscopes are limited by diffraction to about 200 nm resolution and useful magnifications below 2000×.

The target nucleic acid may be imaged with an electron microscope. Exemplary electron microscopes include a transmission electron microscope, a scanning electron microscope, a reflection electron microscope, a scanning transmission electron microscope, a serial blockface scanning electron microscope, a multi-tilt electron microscope, or a cryo-electron microscope as are known in the art.

According to certain aspects, the electron density of the target nucleic acid may be increased and the target nucleic acid may then be imaged. The electron density of the target nucleic acid may be increased by including or adding to the target nucleic acid an electron dense compound. Such electron dense compounds may be added by staining the polymer associated or fixed or complexed with the target nucleic acid with the electron dense compound. Exemplary electron dense compounds include OsO₄, miniSOG, or tetracysteine motif bound to ReAsH (TC/ReAsH). Accordingly, methods include depositing polymers onto a target genome, staining the polymer with an electron dense compound that can be induced in the presence of fluorophores, dyes, other moieties, and/or enzymes to generate oxygen singlets (O₂) and then imaged.

According to one exemplary aspect, electron density of a target nucleic acid can be increased by introducing modified nucleotides into the genome of cells and then chemically attaching polymerization initiators to the modified nucleotides. Suitable methods of chemically attaching are known to those of skill in the art and include common click chemistry reactants known to those of skill in the art, such as azide-functionalized derivatives. The click concept uses a highly capable, small set of chemical reactions that are characterized by high efficiency and yield, orthogonality with other reactions, readily obtained starting materials, stereospecificity, and a robustness that enables them to proceed rapidly. See H. C. Kolb, M. G. Finn and K. B. Sharpless, Angew. Chem., Int. Ed., 2001, 40, 2004 hereby incorporated by reference in its entirety. Monomers are then provided at or near the target nucleic acid. The polymerization initiators are then activated and the monomers are polymerized into a polymer that is complexed with the target nucleic acid. The electron density of the polymer is then increased by the addition of an electron dense compound and the target nucleic acid is then imaged, such as with an electron microscope.

In certain exemplary embodiments, images of Oligopaints, DNA nanostructures, or electron dense moieties hybridized to a target nucleic acid sequence according to the present disclosure are detected and recorded using a computerized imaging system such as the Applied Imaging Corporation CytoVision System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al., supra.

### Electron Dense Compounds as Detectable Moieties

According to one aspect, electron dense compounds attached to the oligopaint can be used as a detectable moiety in an in situ method or otherwise can be used to facilitate detection of the target nucleic acid. According to one aspect, a method of imaging a target nucleic acid sequence in situ includes hybridizing a plurality of oligopaints to the target nucleic acid sequence, wherein each oligopaint of the plurality includes a complementary nucleic acid sequence and a first non-genomic nucleic acid sequence, wherein the first non-genomic nucleic acid sequence includes an electron dense moiety attached thereto, and imaging the target nucleic acid sequence with the oligopaints hybridized thereto. According to one aspect, oligopaint designs utilizing first and second nongenomic sequences as described herein may be used. According to one aspect, polymerization initiators and monomers as discussed herein may be used to create a polymer attached, fixed or complexed to the target nucleic acid sequence as described herein. According to one aspect, the electron density of the polymer may be increased as discussed herein.

In certain embodiments, the detectable moieties can provide higher detectability when used with an electron microscope, compared with common nucleic acids. Moieties with higher detectability are often in the group of metals and organometals, such as mercuric acetate, platinum dimethylsulfoxide, several metal-bipyridyl complexes (e.g. osmium-bipy, ruthenium-bipy, platinum-bipy). While some of these moieties can readily stain nucleic acids specifically, linkers can also be used to attach these moieties to a nucleic acid. Such linkers include acrydite- and a thiol-modified entities, amine reactive groups, and azide and alkyne groups for performing click chemistry. An exemplary electron dense moiety is gold, platinum, silver, uranium, lead, tungsten, lead citrate, sodium phosphotungstate, phosphotungstic acid, or osmium tetroxide. As is known in the art, metallic silver or gold particles may be used to enhance signal from fluorescently labeled nucleotide and/or oligonucleotide sequences (Lakowicz et al. (2003) BioTechniques 34:62).

According to one aspect, some nucleic acid analogs are also more detectable such as gamma-adenosine-thiotriphosphate, iododeoxycytidine-triphosphate, and matellonucleosides in general (see Dale et al., Proc. Nat. Acad. Sci. USA, Vol. 70, No. 8, pp. 2238-2242 (1973)). The modified nucleotides which can be a nucleic acid analog, or a nucleic acid modified with a detectable moiety, or with an attachment chemistry linker, may be added during cell growth.

According to one aspect, a scanning instrument as described herein can be used to visualize and distinguish a target nucleic acid sequence with a detectable moiety. In an exemplary embodiment, the scanning instrument is an electron microscope. Exemplary electron microscopes include a transmission electron microscope (TEM), a scanning electron microscope (SEM), a scanning transmission electron microscope (STEM), and environmental scanning electron microscope (ESEM), a cryo-electron microscope (cryo-EM) and other electron microscopes known to those of skill in the art which can be used to identify local DNA conformation. Such electron microscopes can be used with any of the embodiments described throughout the present disclosure. Transmission electron microscopes and methods of using TEMs are known to those of skill in the art. See Morel, “Visualization of Nucleic Acids,” The Spreading of Nucleic Acids, p. 35-56, CRC Press, Boca Raton (1995) hereby incorporated by reference in its entirety. According to one aspect, the target nucleic acid sequence with the hybridized oligonucleotide probes, i.e oligopaints, with the detectable moieites are visible to the nanometer scale. The EM scanning system scans along a nucleic acid sequence to image the target nucleic acid sequence and the hybridized oligopaints with the detectable moieties. According to one aspect, image processing, edge detection, and object recognition algorithms (such as the Sobel algorithm) can be used to detect the end points and direction vector of the nucleic acid sequence, and inform the motion of the stage. The construct of the target nucleic acid sequence and the hybridized oligonucleotide probes with the detectable moieites may be stained with electron dense compounds such as a heavy metal for EM imaging.

### DNA Nanostructures As Detectable Moieties

According to one aspect, DNA nanostructures attached to the oligopaint can be used as a detectable moieties in an in situ method or otherwise can be used to facilitate detection of the target nucleic acid. According to one aspect, a method of imaging a target nucleic acid sequence in situ includes hybridizing a plurality of oligopaints to the target nucleic acid sequence, wherein each oligopaint of the plurality includes a complementary nucleic acid sequence and a first non-genomic nucleic acid sequence, wherein the first non-genomic nucleic acid sequence includes a DNA nanostructure attached thereto, polymerizing monomers in the presence of a polymerization initiator to create a polymer fixed to the target nucleic acid sequence, and imaging the target nucleic acid sequence with the polymer fixed thereto to detect the DNA nanostructure. According to one aspect, oligopaint designs utilizing first and second nongenomic sequences as described herein may be used. According to one aspect, polymerization initiators and monomers as discussed herein may be used to create a polymer attached, fixed or complexed to the target nucleic acid sequence as described herein. According to one aspect, the electron density of the polymer may be increased as discussed herein.

According to a certain aspect, DNA origami and/or nanostructures may be attached or conjugated to the oligopaint. DNA nanostructure engineering, including multi-strand technologies and DNA origami, can produce an unlimited number of structures, ranging from simple to complex. Further, the structures produced can vary in size from a few to hundreds of nanometers. Based largely on canonical Watson-Crick base pairing, these structures can be designed relatively quickly, using tools such as caDNAno11 and Daedalus12, to function as scaffolds for the precise positioning of proteins, metals, and other nanoparticles, and in some cases facilitating enzymatic activities, drug delivery, and biosensing. According to one aspect, the DNA nanostructure is configured to bind to a cognate binding partner. According to another aspect, the DNA nanostructure is configured to bind to a cognate binding partner such as an aptamer, an antibody, an antigen, or an enzyme. The DNA nanostructures can be present at or within the nongenomic nucleic acid sequence or sequences of the oligopaint as described herein or at or within the complementary or genomic nucleic acid sequence of the oligopaint as described herein.

Aspects of the present disclosure include the use of nucleic acid origami structures. Nucleic acid origami structures, also referred to as DNA origami structures or DNA origami, are two dimensional or three dimensional arbitrary shapes formed from nucleic acids. The DNA origami may be non-naturally occurring nucleic acid nanostructures of arbitrary two dimensional or three dimensional shape. In general, a non-naturally occurring nucleic acid nanostructure of arbitrary two dimensional or three dimensional shape can be made by folding a single stranded nucleic acid scaffold into a custom shape and using oligonucleotide strands to hybridize with the folded single stranded nucleic acid scaffold and hold it into a custom shape. The structure of a DNA origami may be any arbitrary structure as desired. The DNA origami may be attached to an oligonucleotide probe such as an oligopaint and may be detected alone based on its structure or when combined with detectable moiety or a cognate binding partner. According to one aspect, the DNA origami structure is spatially distinct. According to one aspect, the DNA origami structure is geometrically distinct. According to one aspect, the DNA origami structure can be directly visualized using methods known to those of skill in the art. According to one aspect, DNA origami may take the form of any desired shape whether two dimensional or three dimensional. The structure of the unique DNA origami may be visually recognizable and therefore may be distinguishable from other unique DNA origami shapes. Methods of making unique DNA origami shapes of arbitrary design or desired design are described in Rothemund, “Folding DNA to Create Nanoscale Shapes and Patterns”, Nature March 2006, p. 297-302, vol. 440; Rothemund, “Design of DNA Origami”, Proceedings of the International Conference of Computer-Aided Design (ICCAD) 2005; and U.S. Pat. No. 7,842,793 each of which are hereby incorporated by reference in its entirety.

According to an additional embodiment, a DNA origami structure may include one or more detectable moieties at one or more locations within or on the DNA origami structure whether directly or indirectly attached. According to one aspect, the visually detectable spatial orientation of the DNA origami or the one or more detectable moieties at one or more locations within or on the DNA origami, or both, can act detectable species, such as for electron microscope techniques. According to an exemplary embodiment, DNA origami may include nongenomic nucleic acid sequences that may hybridized with complementary nongenomic nucleic acid sequences. Additionally, DNA origami may be tagged with metal nano-particles or fluorophores to enhance distinguishability when analyzed or imaged. Additionally, DNA origami may be tagged with metal nano-particles or fluorophores at distinct locations to enhance distinguishability when analyzed or imaged. Additionally, DNA origami may be tagged with polymerization initiators which may be activated to initiate polymerization of monomers to form a polymer.

According to one aspect, a scanning instrument as described herein can be used to visualize and distinguish nucleic acid origami structures. In an exemplary embodiment, the scanning instrument is an electron microscope. Exemplary electron microscopes include a transmission electron microscope (TEM), a scanning electron microscope (SEM), a scanning transmission electron microscope (STEM), and environmental scanning electron microscope (ESEM), a cryo-electron microscope (cryo-EM) and other electron microscopes known to those of skill in the art which can be used to identify local DNA conformation. Such electron microscopes can be used with any of the embodiments described throughout the present disclosure. Transmission electron microscopes and methods of using TEMs are known to those of skill in the art. See Morel, “Visualization of Nucleic Acids,” The Spreading of Nucleic Acids, p. 35-56, CRC Press, Boca Raton (1995) hereby incorporated by reference in its entirety. According to one aspect, the target nucleic acid sequence with the hybridized oligonucleotide probes, i.e oligopaints, with the DNA origami motifs are visible to the nanometer scale. The EM scanning system scans along a nucleic acid sequence to image the target nucleic acid sequence and the hybridized oligopaints with the DNA origami motifs. According to one aspect, image processing, edge detection, and object recognition algorithms (such as the Sobel algorithm) can be used to detect the end points and direction vector of the nucleic acid sequence, and inform the motion of the stage. The construct of the target nucleic acid sequence and the hybridized oligonucleotide probes with the DNA origami motifs may be stained with electron dense compounds such as a heavy metal for EM imaging.

According to additional aspects, the construct of the nucleic acid template and the hybridized oligopaints with the DNA origami motifs or detectable moiety may be analyzed by methods known to those of skill in the art including high spatial resolution microscopy or super resolution microscopy such as stochastic optical reconstruction microscopy (STORM). Other stochastic methods include spectral precision distance microscopy (SPDM), photoactivated localization microscopy (PALM). Additional methods include deterministic methods such as stimulated emission depletion (STED), ground state depletion (GSD) and spatially structured illumination microscopy (SSIM). Still additional methods include scanning probe microscopy such as atomic force microscopy or scanning tunneling microscopy (STM), as well as, magnetic particles and a magnetic pickup, similar to a hard disk drive head.

According to certain aspects of the present disclosure, a nucleic acid origami structure is a two dimensional structure or a three dimensional structure which is created from DNA. The terms spatially distinct nucleic acid structure, geometrically distinct nucleic acid structure, spatially resolvable nucleic acid structure, spatially observable nucleic acid structure are intended to include the term DNA origami. DNA origami may be a megadalton-scale DNA nanostructure created from one or more or a plurality of DNA strands. According to an exemplary aspect, a nucleic acid origami structure is created from a scaffold strand of a nucleic acid, such as DNA, which is arranged into a desired macromolecular object of a custom shape. Staples strands of DNA, which may be shorter than the scaffold strand of DNA, can be used to direct the folding or other orientation of the scaffold strand of DNA into a programmed arrangement. The term “origami” infers that one or more strands or building blocks of DNA may be folded or otherwise positioned into a desired structure or shape. The desired structure or shape which may then be secured into a desired shape or structure by one or more other strands or building blocks of DNA, such as a plurality of staple strands of DNA. Methods of making DNA origami are known to those of skill in the art. Representative methods include Rothemund, “Folding DNA to Create Nanoscale Shapes and Patterns”, Nature March 2006, p. 297-302, vol. 440; Rothemund, “Design of DNA Origami”, Proceedings of the International Conference of Computer-Aided Design (ICCAD) 2005; U.S. Pat. No. 7,842,793; Douglas et al., Nuc. Acids Res., vol. 37, no. 15, pp. 5001-5006; and Douglas et al., Nature, 459, pp. 414-418 (2009); Andersen et al., Nature, 459, pp. 73-76 (2009); Deitz et al., Science, 325, pp. 725-730 (2009); Han et al., Science, 332, pp. 342-346 (2011); Liu et al., Angew. Chem. Int. Ed., 50, pp. 264-267 (2011); Zhao et al., Nano Lett., 11, pp. 2997-3002 (2011); Woo et al., Nat. Chem. 3, pp. 620-627 (2011) Torring et al., Chem. Soc. Rev. 40, pp. 5636-5646 (2011) each of which are hereby incorporated by reference in their entireties. According to an exemplary aspect, a nucleic acid origami structure need not be constructed of a scaffold strand and staple strands. A nucleic acid origami structure can be constructed by single stranded nucleic acid sequences which self-assemble into tiles to form lattices of any desired shape or size. Such single stranded nucleic acid sequences may be de novo designed and synthesized. Such approaches include programmed self-assembly of such designed strands of nucleic acids to create a wide range of structures with desired shapes. See Wei et al., Nature, volume 485, pp. 623-627 (2012) hereby incorporated by reference in its entirety.

It is to be understood that the principles of the present disclosure do not rely on any particular method of making DNA origami or any particular two dimensional or three dimensional nucleic acid shape. It is to be understood that aspects of the ability of DNA origami to provide unique shapes, to provide locations to hybridize a nucleic acid sequence bearinga functional moiety or group or have directly labeled or tagged functional or detectable moieties is useful in the present methods. It is to be further understood that aspects of the ability to design DNA origami with desired hybridization sites or desired probes is useful in the present methods. It is to be further understood that the ability of DNA origami to be of sufficient size to be identified by visualizing the shape of the DNA origami is in the present methods. It is to be further understood that the ability of DNA origami to be of sufficient size to be directly visually distinguishable is useful in the present methods. It is to be further understood that the ability of DNA origami to be megadalton-scale nucleic acid (such as DNA) nanostructures of sufficient size to be identified by visualizing the shape of the DNA origami is in the present methods. According to certain aspects of the present disclosure, a nucleic acid origami structure is attached to an oligonucleotide probe such as an oligopaint. The nucleic acid origami structure may include a detectable moiety, label, reporter or polymerization initiator. The nucleic acid origami structure may include a probe hybridization site for hybridizing with a probe having a detectable moiety, label, reporter or polymerization initiator. This concept may be referred to as indirect attachment as described herein. The nucleic acid origami structure may have a geometrically distinct or geometrically unique structure. Methods of making nucleic acid origami structures are known to those of skill in the art. Methods of attaching a detectable moiety, label, reporter or polymerization initiator to a nucleic acid sequence are known to those of skill in the art.

The contents of all references, patents and published patent applications cited throughout this application are hereby incorporated by reference in their entirety for all purposes.

### EXAMPLE I

### Measuring the Density and Distance Between Genomic Targets

According to one aspect, a target genomic nucleic acid sequence is desired to be detected or visualized in situ using an electron microscope, such as to achieve 3 to 5 nanometer resolution and sequence specificity. Oligopaints are designed as described herein such that they will hybridize to the target genomic nucleic acid sequence. The sequence of the oligopaints are designed so as to have a desired determined density, i.e. number of oligopaints to be hybridized to the target genomic nucleic acid sequence, so as to generate a signal sufficient for detection. The oligopaints have a detectable moiety attached thereto, or a DNA nanostructure or a polymerization initiator, as described herein. Polymerization initiators, such as dyes capable of photo-inducing DAB polymerization may be used.

According to one aspect, when targeting single copy regions, there are two signals per nucleus. By targeting two closely linked loci, the signals will be near each other, and by targeting loci of different sizes, corresponding shifts in signal size are obtained. In terms of resolution, a series of DNA nanostructures that place dyes in repeating patterns may be designed, such that the series will span dye-to-dye distances of 5 to 20 nm. This strategy will allow the comparing of images from different sections of the same nanostructure to assess efficacy, resolution, and reproducibility. The DNA nanostructures, if used, may include DNA origami and/or nanostructures that generate an aptamer or binding site for antibodies, enzymes, enzymatic activity, ligand, etc. According to one aspect, multiple DNA origami and/or nanostructures may be positioned on a single oligopaint, such as at or within the nongenomic sequence or sequences or at or within the complementary genomic sequence, so as to facilitate direct interactions between the different structures.

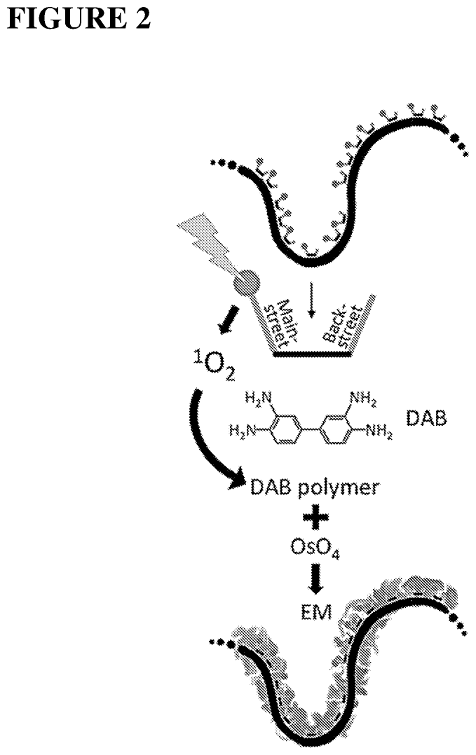

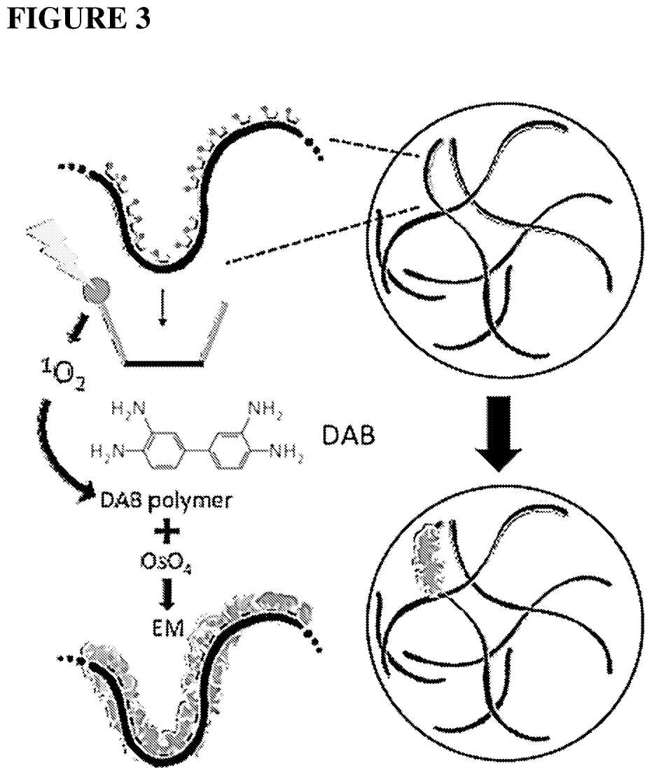

Embodiments of the current disclosure include “click chemistry” techniques that expand the current capabilities of electron microscopy by depositing polymers onto a target genome, staining the polymer with an electron dense compound that can be induced in the presence of fluorophores, dyes, other moieties, and/or enzymes to generate oxygen singlets (O₂) and then imaged. For example, electron microscopy-level genome imaging may include growing cells in 5-ethynyl-2′-deoxyuridine (EdU), fixing the cells, subjecting the cells to click chemistry in the presence of azide-functionalized derivatives of dyes, such that EdU residues are coupled to the dyes, subjecting the cells to intense illumination to generate oxygen singlets (O₂) that, in the presence of 3,3′-diaminobenzydene (DAB), induce DAB polymerization, staining the polymers with OsO₄ to render them electron dense, embedding the sample in resin (e.g., Durcupan ACM, Electron Microscopy Sciences), sectioning the sample (e.g., with a microtome), and then imaging the sample with transmission electron microscopy. As shown in FIGS. 2 and 3, a target genome within a cell is “stained” with a fluorescent dye (e.g., eosin, methylene blue) attached to a nongenomic nucleic acid sequence of an oligopaint which hybridizes at a plurality of locations along the target nucleic acid sequence. The cell is incubated in 3,3′-diaminobenzydene (DAB) and exposed to illumination, i.e. light of desired wavelength. The 3,3′-diaminobenzydene (DAB) is converted into an osmiophilic polymer in the presence of the oxygen singlets (O₂) released when the dye is exposed to illumination. Further staining of the polymer with OsO₄ enables the target genome to be imaged by EM.

According to other aspects, the method of imaging a target nucleic acid sequence in situ further includes increasing the electron density of the target nucleic acid sequence with the polymer fixed thereto by staining the polymer with an electron dense compound, and imaging the electron dense target nucleic acid sequence with the polymer fixed thereto. According to one aspect, the method of imaging a target nucleic acid sequence in situ further includes increasing electron density of the target nucleic acid sequence with the polymer fixed thereto by staining the polymer with 0s04, miniSOG, or tetracysteine motif bound to ReAsH (TC/ReAsH) and imaging the electron dense target nucleic acid sequence with the polymer fixed thereto. According to one aspect, the methods described in the current disclosure achieve sequence-specific electron microscope imaging by targeting detectable moiety or dye-coupled or DNA nanostructure containing oligopaints hybridized to sub-regions of the genome. Illumination results in localized deposition of DAB polymers, conferring sequence-specificity to electron microscope imaging. In order to image entire nuclei that can have depths of >15 μm, automated serial block-face ion beam tomography, capable of 1 nm steps in z, and multi-tilt EM tomography can be used as well as highly coordinated strategies for data/image collection, alignment, and processing.

### Embodiments

The present disclosure provides a method of imaging a target nucleic acid sequence in situ including hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes a polymerization initiator attached thereto, activating the polymerization initiator in the presence of monomers to initiate polymerization of the monomers to create a polymer fixed to the target nucleic acid sequence, and imaging the target nucleic acid sequence with the polymer fixed thereto. According to one aspect, the method further includes increasing electron density of the target nucleic acid sequence with the polymer fixed thereto, and imaging the electron dense target nucleic acid sequence with the polymer fixed thereto. According to one aspect, the method further includes increasing electron density of the target nucleic acid sequence with the polymer fixed thereto by staining the polymer with an electron dense compound, and imaging the electron dense target nucleic acid sequence with the polymer fixed thereto. According to one aspect, the method further includes increasing electron density of the target nucleic acid sequence with the polymer fixed thereto by staining the polymer with OsO₄, miniSOG, or TC/ReAsH and imaging the electron dense target nucleic acid sequence with the polymer fixed thereto. According to one aspect, the first non-genomic nucleic acid sequence is upstream of the complementary nucleic acid sequence, and wherein the Oligopaint further includes a second non-genomic nucleic acid sequence downstream of the complementary nucleic acid sequence. According to one aspect, the method further includes separating the target nucleic acid sequence into an upper strand and a lower strand, andhybridizing the Oligopaint to the upper strand or the lower strand. According to one aspect, the monomers are aromatic amino monomers that polymerize through initiation by singlet oxygen. According to one aspect, the polymer is formed by polymerization of aromatic amino monomers that polymerize through initiation by singlet oxygen. According to one aspect, the target nucleic acid species is genomic DNA, RNA, single-copy DNA, repeated DNA, in situ DNA, in vitro DNA, cDNA, synthetic DNA, antibodies with a nucleic acid tail, or combinations thereof. According to one aspect, the target nucleic acid sequence with the polymer fixed thereto is imaged with an electron microscope. According to one aspect, the target nucleic acid sequence with the polymer fixed thereto is imaged with an electron microscope such as a transmission electron microscope, a scanning electron microscope, a reflection electron microscope, a scanning transmission electron microscope, a serial blockface scanning electron microscope, a multi-tilt electron microscope, or a cryo-electron microscope. According to one aspect, the polymerization initiator is a photoinduced polymerization initiator. According to one aspect, the polymerization initiator is a photoinduced polymerization initiator and polymerization is induced with a laser. According to one aspect, the polymerization initiator generates oxygen singlets to induce polymerization of the monomers. According to one aspect, the polymerization initiator is a dye or a fluorophore. According to one aspect, the polymerization initiator is a dye or a fluorophore such as fluorescein, dibromofluorescein (DBF), eosin, tetramethylrhodamine (TAMRA), monobromo-TAMRA (Br-TAMRA), AlexaFluor 488 (AF488), AlexaFluor 633 (AF633), monobromo-Cy5 (Br-Cy5), methylene blue (MB), or IRDye700DX. According to one aspect, the polymerization initiator is directly attached to the first non-genomic nucleic acid sequence. According to one aspect, the polymerization initiator is indirectly attached to the first non-genomic nucleic acid sequence.

The present disclosure provides a method of imaging a target nucleic acid sequence in situ including hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes a DNA nanostructure attached thereto, polymerizing monomers in the presence of a polymerization initiator to create a polymer fixed to the target nucleic acid sequence, and imaging the target nucleic acid sequence with the polymer fixed thereto to detect the DNA nanostructure. According to one aspect, the method further includes increasing electron density of the target nucleic acid sequence with the polymer fixed thereto, and imaging the electron dense target nucleic acid sequence with the polymer fixed thereto to detect the DNA nanostructure. According to one aspect, the method further includes increasing electron density of the target nucleic acid sequence with the polymer fixed thereto by staining the polymer with an electron dense compound, and imaging the electron dense target nucleic acid sequence with the polymer fixed thereto to detect the DNA nanostructure. According to one aspect, the method further includes increasing electron density of the target nucleic acid sequence with the polymer fixed thereto by staining the polymer with OsO₄, miniSOG, or TC/ReAsH and imaging the electron dense target nucleic acid sequence with the polymer fixed thereto to detect the DNA nanostructure. According to one aspect, the first non-genomic nucleic acid sequence is upstream of the complementary nucleic acid sequence, and wherein the Oligopaint further includes a second non-genomic nucleic acid sequence downstream of the complementary nucleic acid sequence. According to one aspect, the method further includes separating the target nucleic acid sequence into an upper strand and a lower strand, and hybridizing the Oligopaint to the upper strand or the lower strand. According to one aspect, the monomers are aromatic amino monomers that polymerize through initiation by singlet oxygen. According to one aspect, the polymer is formed by polymerization of aromatic amino monomers that polymerize through initiation by singlet oxygen. According to one aspect, the target nucleic acid species is genomic DNA, RNA, single-copy DNA, repeated DNA, in situ DNA, in vitro DNA, cDNA, synthetic DNA, antibodies with a nucleic acid tail, or combinations thereof. According to one aspect, the target nucleic acid sequence with the polymer fixed thereto is imaged with an electron microscope. According to one aspect, the target nucleic acid sequence with the polymer fixed thereto is imaged with an electron microscope such as a transmission electron microscope, a scanning electron microscope, a reflection electron microscope, a scanning transmission electron microscope, a serial blockface scanning electron microscope, a multi-tilt electron microscope, or a cryo-electron microscope. According to one aspect, the polymerization initiator is a photoinduced polymerization initiator. According to one aspect, the polymerization initiator is a photoinduced polymerization initiator and polymerization is induced with a laser. According to one aspect, the polymerization initiator generates oxygen singlets to induce polymerization of the monomers. According to one aspect, the polymerization initiator is a dye or a fluorophore. According to one aspect, the polymerization initiator is a dye or a fluorophore such as fluorescein, dibromofluorescein (DBF), eosin, tetramethylrhodamine (TAMRA), monobromo-TAMRA (Br-TAMRA), AlexaFluor 488 (AF488), AlexaFluor 633 (AF633), monobromo-Cy5 (Br-Cy5), methylene blue (MB), or IRDye700DX. According to one aspect, the DNA nanostructure is directly attached to the first non-genomic nucleic acid sequence. According to one aspect, the DNA nanostructure is indirectly attached to the first non-genomic nucleic acid sequence. According to one aspect, the DNA nanostructure is configured to bind to a cognate binding partner. According to one aspect, the DNA nanostructure is configured to bind to a cognate binding partner such as an aptamer, an antibody, an antigen, or an enzyme.

The present disclosure provides a method of imaging a target nucleic acid sequence in situ including hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes an electron dense moiety attached thereto, and imaging the target nucleic acid sequence with the Oligopaints hybridized thereto. According to one aspect, the electron dense moiety is gold, platinum, silver, uranium, lead, tungsten, lead citrate, sodium phosphotungstate, phosphotungstic acid, or osmium tetroxide. According to one aspect, the first non-genomic nucleic acid sequence is upstream of the complementary nucleic acid sequence, and wherein the Oligopaint further includes a second non-genomic nucleic acid sequence downstream of the complementary nucleic acid sequence. According to one aspect, the method further includes separating the target nucleic acid sequence into an upper strand and a lower strand, and hybridizing the Oligopaint to the upper strand or the lower strand. According to one aspect, the target nucleic acid species is genomic DNA, RNA, single-copy DNA, repeated DNA, in situ DNA, in vitro DNA, cDNA, synthetic DNA, antibodies with a nucleic acid tail, or combinations thereof. According to one aspect, the target nucleic acid sequence with the plurality of Oligonucleotide paints hybridized thereto is imaged with an electron microscope. According to one aspect, the target nucleic acid sequence with the plurality of Oligonucleotide paints hybridized thereto is imaged with an electron microscope such as a transmission electron microscope, a scanning electron microscope, a reflection electron microscope, a scanning transmission electron microscope, a serial blockface scanning electron microscope, a multi-tilt electron microscope, or a cryo-electron microscope. According to one aspect, the electron dense moiety is directly attached to the first non-genomic nucleic acid sequence. According to one aspect, the electron dense moiety is indirectly attached to the first non-genomic nucleic acid sequence.

### Equivalents

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. Other embodiments will be evident to those of skill in the art. It should be understood that the foregoing description is provided for clarity only and is merely exemplary. All publications, patents and patent applications cited above are incorporated by reference herein in their entirety for all purposes to the same extent as if each individual publication or patent application were specifically indicated to be so incorporated by reference.

### REFERENCES

References identified herein and listed as follows are hereby incorporated by reference herein in their entireties for all purposes. The references identified below may be referred to herein by the number associated with the reference.
1. Beliveau B J, Joyce E F, Apostolopoulos N, Yilmaz F, Fonseka C Y, McCole R B, Chang Y, Li J B, Senaratne T N, Williams B R, Rouillard J M, Wu C T. Versatile design and synthesis platform for visualizing genomes with Oligopaint FISH probes. Proc Natl Acad Sci USA. 2012 109:21301-6. PMID: 23236188; PMCID: PMC3535588.
2. Beliveau B J, Apostolopoulos N, Wu C T. Visualizing genomes with Oligopaint FISH probes. Curr Protoc Mol Biol. 2014 105:Unit 14.23. PMID: 24510436 PMCID: PMC3928790.
3. Beliveau B J, Boettiger A N, Avendaño M S, Jungmann R, McCole R B, Joyce E F, Kim-Kiselak C, Bantignies F, Fonseka C, Erceg J, Hannan M, Hoang H, Colognori D, Lee J T, Shih W M, Yin P, Zhuang X, Wu C T. Single-molecule super-resolution imaging of chromosomes and in situ haplotype visualization using Oligopaint FISH probes Nat. Commun. 2015 6:7147 PMID: 25962338 PMCID: PMC4430122.
4. Ngo J T, Adams S R, Deerinck T J, Boassa D, Rodriguez-Rivera F, Palida S F, Bertozzi C R, Ellisman M H, Tsien R Y. Click-E M for imaging metabolically tagged nonprotein biomolecules. Nat Chem Biol. 2016 12:459-65. doi: 10.1038/nchembio.2076. Epub 2016 Apr. 25. PMID: 27110681 PMCID: PMC4871776.
5. Maranto A R. Neuronal mapping: a photooxidation reaction makes Lucifer yellow useful for electron microscopy. Science. 1982 Sep. 3; 217(4563):953-5. PMID: 7112109.
6. Deerinck T J, Martone M E, Lev-Ram V, Green D P, Tsien R Y, Spector D L, Huang S, Ellisman M H. Fluorescence photooxidation with eosin: a method for high resolution immunolocalization and in situ hybridization detection for light and electron microscopy. J Cell Biol. 1994 August; 126(4):901-10. PMID: 7519623 PMCID: PMC2120127.
7. Shu X, Lev-Ram V, Deerinck T J, Qi Y, Ramko E B, Davidson M W, Jin Y, Ellisman M H, Tsien R Y. A genetically encoded tag for correlated light and electron microscopy of intact cells, tissues, and organisms. PLoS Biol. 2011 April; 9(4):e1001041. doi: 10.1371/journal.pbio.1001041. Epub 2011 Apr. 5. PMID: 21483721 PMCID: PMC3071375.
8. Jabbari H, Aminpour M, Montemagno C. Computational Approaches to Nucleic Acid Origami. ACS Comb Sci. 2015 Oct. 12; 17(10):535-47. doi: 10.1021/acscombsci.5b00079. Epub 2015 Sep. 17. PMID: 26348196.
9. Chandrasekaran A R, Anderson N, Kizer M, Halvorsen K, Wang X. Beyond the Fold: Emerging Biological Applications of DNA Origami. Chembiochem. 2016 Jun. 16; 17(12):1081-9. doi: 10.1002/cbic.201600038. Epub 2016 Apr. 6. PMID: 26928725.
10. Pfeifer W, Saccà B. From Nano to Macro through Hierarchical Self-Assembly: The DNA Paradigm. Chembiochem. 2016 Jun. 16; 17(12):1063-80. doi: 10.1002/cbic.201600034. Epub 2016 May 17. PMID: 27186937.
11. Douglas S M, Marblestone A H, Teerapittayanon S, Vazquez A, Church G M, Shih W M. Rapid prototyping of 3D DNA-origami shapes with caDNAno. Nucleic Acids Res. 2009 August; 37(15):5001-6. doi: 10.1093/nar/gkp436. Epub 2009 Jun. 16. PMID: 19531737 PMCID: PMC2731887.
12. Veneziano R, Ratanalert S, Zhang K, Zhang F, Yan H, Chiu W, Bathe M. Designer nanoscale DNA assemblies programmed from the top down. Science. 2016 Jun. 24; 352(6293):1534. doi: 10.1126/science.aaf4388. Epub 2016 May 26. PMID: 27229143 PMCID: PMC5111087.

What is claimed is:

1. A method of imaging a target nucleic acid sequence in situ comprising hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes a polymerization initiator attached thereto,
activating the polymerization initiator in the presence of monomers to initiate polymerization of the monomers to create a polymer fixed to the target nucleic acid sequence, and
imaging the target nucleic acid sequence with the polymer fixed thereto.

2. The method of claim 1 further comprising increasing electron density of the target nucleic acid sequence with the polymer fixed thereto, and imaging the electron dense target nucleic acid sequence with the polymer fixed thereto.

3. The method of claim 1 further comprising increasing electron density of the target nucleic acid sequence with the polymer fixed thereto by staining the polymer with an electron dense compound, and imaging the electron dense target nucleic acid sequence with the polymer fixed thereto.

4. The method of claim 1 further comprising increasing electron density of the target nucleic acid sequence with the polymer fixed thereto by staining the polymer with 0s04, miniSOG, or TC/ReAsH and imaging the electron dense target nucleic acid sequence with the polymer fixed thereto.

5. The method of claim 1 wherein the first non-genomic nucleic acid sequence is upstream of the complementary nucleic acid sequence, and wherein the Oligopaint further includes a second non-genomic nucleic acid sequence downstream of the complementary nucleic acid sequence.

6. The method of claim 1 further comprising separating the target nucleic acid sequence into an upper strand and a lower strand, and
hybridizing the Oligopaint to the upper strand or the lower strand.

7. The method of claim 1 wherein the monomers are aromatic amino monomers that polymerize through initiation by singlet oxygen.

8. The method of claim 1 wherein the polymer is formed by polymerization of aromatic amino monomers that polymerize through initiation by singlet oxygen.

9. The method of claim 1 wherein the target nucleic acid species is genomic DNA, RNA, single-copy DNA, repeated DNA, in situ DNA, in vitro DNA, cDNA, synthetic DNA, antibodies with a nucleic acid tail, or combinations thereof.

10. The method of claim 1 wherein the target nucleic acid sequence with the polymer fixed thereto is imaged with an electron microscope.

11. The method of claim 1 wherein the target nucleic acid sequence with the polymer fixed thereto is imaged with an electron microscope selected from the group consisting of a transmission electron microscope, a scanning electron microscope, a reflection electron microscope, a scanning transmission electron microscope, a serial blockface scanning electron microscope, a multi-tilt electron microscope, or a cryo-electron microscope.

12. The method of claim 1 wherein the polymerization initiator is a photoinduced polymerization initiator.

13. The method of claim 1 wherein the polymerization initiator is a photoinduced polymerization initiator and polymerization is induced with a laser.

14. The method of claim 1 wherein the polymerization initiator generates oxygen singlets to induce polymerization of the monomers.

15. The method of claim 1 wherein the polymerization initiator is a dye or a fluorophore.

16. The method of claim 1 wherein the polymerization initiator is a dye or a fluorophore selected from the group consisting of fluorescein, dibromofluorescein (DBF), eosin, tetramethylrhodamine (TAMRA), monobromo-TAMRA (Br-TAMRA), AlexaFluor 488 (AF488), AlexaFluor 633 (AF633), monobromo-Cy5 (Br-Cy5), methylene blue (MB), or IRDye700DX.

17. The method of claim 1 wherein the polymerization initiator is directly attached to the first non-genomic nucleic acid sequence.

18. The method of claim 1 wherein the polymerization initiator is indirectly attached to the first non-genomic nucleic acid sequence.

19. A method of imaging a target nucleic acid sequence in situ comprising hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes a DNA nanostructure attached thereto,
polymerizing monomers in the presence of a polymerization initiator to create a polymer fixed to the target nucleic acid sequence, and
imaging the target nucleic acid sequence with the polymer fixed thereto to detect the DNA nanostructure.

20. The method of claim 19 further comprising increasing electron density of the target nucleic acid sequence with the polymer fixed thereto, and imaging the electron dense target nucleic acid sequence with the polymer fixed thereto to detect the DNA nanostructure.

21. The method of claim 19 further comprising increasing electron density of the target nucleic acid sequence with the polymer fixed thereto by staining the polymer with an electron dense compound, and imaging the electron dense target nucleic acid sequence with the polymer fixed thereto to detect the DNA nanostructure.

22. The method of claim 19 further comprising increasing electron density of the target nucleic acid sequence with the polymer fixed thereto by staining the polymer with 0s04, miniSOG, or TC/ReAsH and imaging the electron dense target nucleic acid sequence with the polymer fixed thereto to detect the DNA nanostructure.

23. The method of claim 19 wherein the first non-genomic nucleic acid sequence is upstream of the complementary nucleic acid sequence, and wherein the Oligopaint further includes a second non-genomic nucleic acid sequence downstream of the complementary nucleic acid sequence.

24. The method of claim 19 further comprising separating the target nucleic acid sequence into an upper strand and a lower strand, and
hybridizing the Oligopaint to the upper strand or the lower strand.

25. The method of claim 19 wherein the monomers are aromatic amino monomers that polymerize through initiation by singlet oxygen.

26. The method of claim 19 wherein the polymer is formed by polymerization of aromatic amino monomers that polymerize through initiation by singlet oxygen.

27. The method of claim 19 wherein the target nucleic acid species is genomic DNA, RNA, single-copy DNA, repeated DNA, in situ DNA, in vitro DNA, cDNA, synthetic DNA, antibodies with a nucleic acid tail, or combinations thereof.

28. The method of claim 19 wherein the target nucleic acid sequence with the polymer fixed thereto is imaged with an electron microscope.

29. The method of claim 19 wherein the target nucleic acid sequence with the polymer fixed thereto is imaged with an electron microscope selected from the group consisting of a transmission electron microscope, a scanning electron microscope, a reflection electron microscope, a scanning transmission electron microscope, a serial blockface scanning electron microscope, a multi-tilt electron microscope, or a cryo-electron microscope.

30. The method of claim 19 wherein the polymerization initiator is a photoinduced polymerization initiator.

31. The method of claim 19 wherein the polymerization initiator is a photoinduced polymerization initiator and polymerization is induced with a laser.

32. The method of claim 19 wherein the polymerization initiator generates oxygen singlets to induce polymerization of the monomers.

33. The method of claim 19 wherein the polymerization initiator is a dye or a fluorophore.

34. The method of claim 19 wherein the polymerization initiator is a dye or a fluorophore selected from the group consisting of fluorescein, dibromofluorescein (DBF), eosin, tetramethylrhodamine (TAMRA), monobromo-TAMRA (Br-TAMRA), AlexaFluor 488 (AF488), AlexaFluor 633 (AF633), monobromo-Cy5 (Br-Cy5), methylene blue (MB), or IRDye700DX.

35. The method of claim 19 wherein the DNA nanostructure is directly attached to the first non-genomic nucleic acid sequence.

36. The method of claim 19 wherein the DNA nanostructure is indirectly attached to the first non-genomic nucleic acid sequence.

37. The method of claim 19 wherein the DNA nanostructure is configured to bind to a cognate binding partner.

38. The method of claim 19 wherein the DNA nanostructure is configured to bind to a cognate binding partner selected from the group consisting of an aptamer, an antibody, an antigen, or an enzyme.

39. A method of imaging a target nucleic acid sequence in situ comprising
hybridizing a plurality of Oligopaints to the target nucleic acid sequence, wherein each Oligopaint of the plurality includes a complementary nucleic acid sequence and a first non-genomic nucleic acid sequence, wherein the first non-genomic nucleic acid sequence includes an electron dense moiety attached thereto, and
imaging the target nucleic acid sequence with the Oligopaints hybridized thereto.

40. The method of claim 39 wherein the electron dense moiety is a member selected from the group consisting of gold, platinum, silver, uranium, lead, tungsten, lead citrate, sodium phosphotungstate, phosphotungstic acid, or osmium tetroxide.

41. The method of claim 39 wherein the first non-genomic nucleic acid sequence is upstream of the complementary nucleic acid sequence, and wherein the Oligopaint further includes a second non-genomic nucleic acid sequence downstream of the complementary nucleic acid sequence.

42. The method of claim 39 further comprising separating the target nucleic acid sequence into an upper strand and a lower strand, and
hybridizing the Oligopaint to the upper strand or the lower strand.

43. The method of claim 39 wherein the target nucleic acid species is genomic DNA, RNA, single-copy DNA, repeated DNA, in situ DNA, in vitro DNA, cDNA, synthetic DNA, antibodies with a nucleic acid tail, or combinations thereof.

44. The method of claim 39 wherein the target nucleic acid sequence with the plurality of Oligonucleotide paints hybridized thereto is imaged with an electron microscope.

45. The method of claim 39 wherein the target nucleic acid sequence with the plurality of Oligonucleotide paints hybridized thereto is imaged with an electron microscope selected from the group consisting of a transmission electron microscope, a scanning electron microscope, a reflection electron microscope, a scanning transmission electron microscope, a serial blockface scanning electron microscope, a multi-tilt electron microscope, or a cryo-electron microscope.

46. The method of claim 39 wherein the electron dense moiety is directly attached to the first non-genomic nucleic acid sequence.

47. The method of claim 39 wherein the electron dense moiety is indirectly attached to the first non-genomic nucleic acid sequence.
